(19) United States
(12) Patent Application Publication
(10) Pub. No.: US 2024/0075108 A1
(43) Pub. Date: Mar. 7, 2024

(54) HEPATOCELLULAR CARCINOMA TREATMENTS, PROPHYLACTIC THERAPIES, AND COMPOSITIONS FOR USE THEREWITH

(71) Applicant: REPAIR BIOTECHNOLOGIES, INC., Syracuse, NY (US)

(72) Inventors: Mourad Topors, Syracuse, NY (US); Reason, Syracuse, NY (US); Guilherme Cherman Perdigao de Oliveira, Syracuse, NY (US); Marc Ridilla, Syracuse, NY (US); David Mackenzie-Liu, Syracuse, NY (US); Garrett Strough, Syracuse, NY (US)

(21) Appl. No.: 18/269,869

(22) PCT Filed: Dec. 29, 2021
(86) PCT No.: PCT/US2021/065430

§ 371 Date: Jun. 27, 2023

Related U.S. Application Data
(60) Provisional application No. 63/131,952, filed on Dec. 30, 2020.

(51) Int. Cl. A61K 38/44; A61K 31/44; A61K 31/47; A61K 38/17; A61K 38/52; A61P 35/00

(52) U.S. Cl. CPC A61K 38/443; A61K 31/44; A61K 31/47; A61K 38/17; A61K 38/52; A61P 35/00; C12Y 101/01146; C12Y 101/01147; C12Y 101/03006; C12Y 103/99004; C12Y 114/14001; C12Y 503/03001

(57) ABSTRACT

The disclosed compositions, methods, and systems are useful in treating and preventing various cancers associated with buildup of mitochondrial cholesterol. In many embodiments, the disclosed compositions, methods, and systems may improve the efficacy and cytotoxicity of chemotherapeutic compounds, for example increasing cellular sensitivity to chemotherapy and/or reducing or preventing resistance. In many embodiments, the cancer may be early or late stage hepatocellular carcinoma.

### CROSS-REFERENCE TO RELATED APPLICATIONS

This application claims benefit of priority pursuant to 35 U.S.C. § 119(e) of U.S. provisional patent application No. 63/131,952 entitled “Hepatocellular Carcinoma Treatments, Prophylactic Therapies, and Compositions for Use Therewith,” filed on 30 Dec. 2020, which is hereby incorporated by reference in its entirety.

### FIELD

The disclosed processes, methods, and systems are directed to therapy for various cancers, including hepatocellular carcinomas.

### SEQUENCE LISTING

The instant application contains a Sequence Listing which has been submitted electronically in ASCII format and is hereby incorporated by reference in its entirety. Said ASCII copy, created on Dec. 27, 2021, is named p289349_WO_01_ST25.txt and is 34,319 bytes in size.

### BACKGROUND

Cholesterol dysregulation has been implicated in genesis, progression, and maintenance of cancer phenotypes. Specifically, mitochondrial cholesterol concentration has been shown to be aberrant in solid tumors and associated with tumor growth, malignancy, chemo-sensitivity, and resistance to therapies. Recently, statin drugs, which target the cholesterol synthesis pathway, have been shown to improve the efficacy and cytotoxicity of some chemotherapeutics. One particular type of cancer that appears to be associated with cholesterol buildup is hepatocellular carcinoma, HCC, and others include pancreatic cancer. While statins may reduce the build-up of cholesterol in cells, they do not directly target cholesterol that has already accumulated with the cells.

What is needed are therapies for treating cancer, for example HCC that are effective, do not induce resistance, and are safer that current therapies.

### SUMMARY

Disclosed herein are compositions and methods useful in treating and/or preventing cancers, including hepatocellular carcinoma, in a subject in need thereof. In many embodiments, the disclosed compositions and methods are useful in treating and/or preventing diseases and conditions that may be associated with alterations in the uptake, trafficking, accumulation and/or metabolism of cholesterol, for example those associated with abnormal cell proliferation and/or survival. In many embodiments, the disclosed methods may include expressing one or more cholesterol catabolizing proteins in at least one liver cell of the subject and reducing the growth rate of the at least one or more liver cell. In many embodiments, the one or more cholesterol catabolizing proteins may be expressed in a mitochondrion of the liver cell. In many embodiments, the methods may also include contacting the liver cell with at least one chemotherapeutic compound, before, after, or while the cell is expressing the one or more cholesterol catabolizing protein, which may be selected from steroidogenic acute regulatory (StAR) protein, cholesterol dehydrogenase (CholD; SEQ ID NO:2), 3-ketosteroid Δ1-dehydrogenase (Δ1-KstD; SEQ ID NO:3), anoxic cholesterol metabolism B enzyme (acmB; SEQ ID NO:4), 3-ketosteroid 9α-hydroxylase (KshAB; SEQ ID NO:5), 3β-hydroxysteroid dehydrogenase 2 (HSD2; SEQ ID NO:6), P450-ferredoxin reductase-ferredoxin fusion protein (P450-FdxR-Fdx; SEQ ID NO:1), and combinations thereof. In many embodiments, the chemotherapeutic compound may be selected from Sorafenib and Lenvatinib, and the method may be useful in preventing or reducing in the likelihood of a mammalian cell being resistant to chemotherapy. In many embodiments, the method may result in the liver cell may enter the apoptotic pathway after expressing the one or more cholesterol catabolizing protein.

Also disclosed are compositions for reducing the growth rate or viability of hepatic cells, especially hepatic carcinoma cells, for example hepatocellular carcinoma cells, the composition comprising, a nucleic acid coding for at least one cholesterol catabolizing protein and a nucleic acid comprising a promoter that is active in a mammalian liver cell, wherein the nucleic acid may be within a cell, viral particle, or lipid nanoparticle. In many embodiments, the composition may also comprise a pharmaceutically acceptable compound or salt, and may be used in the making of a medicament for the treatment or prevention of liver disease or cancer. In many embodiments, the at least one cholesterol catabolizing proteins are selected from steroidogenic acute regulatory (StAR) protein, cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx), and combinations thereof.

Also disclosed are methods of treating or preventing hepatocellular carcinoma in a subject in need thereof comprising: administering a composition to the subject, wherein the composition comprises one or more compounds that support expression of at least one cholesterol catabolizing protein in a mitochondrion of a mammalian cell. In many embodiments, the subject may also be administered a chemotherapeutic compound before, after, or with the composition, and the chemotherapeutic compound may comprise Sorafenib or Lenvatinib. In many embodiments, the at least one cholesterol catabolizing proteins may be selected from steroidogenic acute regulatory (StAR) protein, cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx), and combinations thereof. In many embodiments the method may be useful in preventing or reducing likelihood of a liver cell being resistant to chemotherapy and/or may result in the liver cell may enter the apoptotic pathway after expressing the one or more cholesterol catabolizing protein.

### BRIEF DESCRIPTION OF THE DRAWINGS

FIG. 1 is a representative western blot showing the expression of Flag-tagged CDP in CDP-Hep3B cells and not in Mock-Hep3B cells.

FIG. 2 is a representative graph showing a reduction in the growth rate of CDP-Hep3B compared with Mock-Hep3B cells over 48 hours

### DETAILED DESCRIPTION

Disclosed herein are compositions, methods, and systems for treating and preventing various diseases and conditions associated with alterations in the cellular uptake, trafficking, accumulation, and/or metabolism of cholesterol, especially those that are associated with abnormal cell proliferation and/or survival. In many embodiments, the disease or condition may be a cancer, for example hepatocellular carcinoma. In many embodiments, the disclosed compositions, methods, and systems are helpful in reducing cholesterol content of one or more liver cells. In many embodiments, the disclosed compositions, methods, and systems may include therapies combining compositions and methods for reducing cellular cholesterol levels and chemotherapeutic compositions and methods. In many embodiments, the disclosed compositions, methods, and systems may be useful in reducing mitochondrial cholesterol content. In many embodiments, the disclosed compositions, methods, and systems may result in one or more cancerous or pre-cancerous cells being more sensitive or less resistant to one or more chemotherapeutic compounds and/or therapies. In many embodiments, the disclosed sensitivity/resistance may be measured by various methods, for example IC₅₀, a quantitative measure that indicates how much of a particular inhibitory substance (here, a chemotherapeutic compound) is able to inhibit cell or cellular characteristic by 50%. In some embodiments, the chemotherapeutic compounds may be selected from Sorafenib or Lenvatinib.

### Hepatocellular Carcinoma (HCC)

Hepatocellular carcinoma (HCC) is one of the deadliest cancers in the world. There are limited treatment options because HCC it is often diagnosed at an advanced stage. While surgical resection is the preferred therapy, only ˜15% of HCC patients are eligible for such intervention, and ˜70% of those patients experience a recurrence within 5 years.

At present, pharmaceutical therapies for treating HCC are very limited. Sorafenib Tosylate (Nexavar from Bayer; a multi-kinase inhibitor, that exerts anti-proliferative (RAF1, BRAF, and KIT), antiangiogenic (vascular endothelial growth factor receptor [VEGFR] and platelet-derived growth factor receptor”[PDGFRB]), and pro-apoptotic effects) and Lenvatinib Mesylate (Lenvima from Merck; multikinase inhibitor of VEGFRs 1-3, fibroblast growth factor receptors (FGFRs) 1-4, RET, KIT, and PDGFRa) are the only approved drugs for first-line systemic treatment of advanced HCC that cannot be removed by surgery. Second-line therapies include the multi-kinase inhibitors, regorafenib and cabozantinib, the anti-VEGFR2 mAb, ramucirumab, and the immune checkpoint inhibitors (Anti-PD-1 mAb), nivolumab and pembrolizumab.

Sorafenib is an orally active multi-kinase inhibitor that has demonstrated significant anti-cancer activity in phase III clinical trials in patients with HCC. However, resistance to Sorafenib is not uncommon, and a limiting factor in the effectiveness of this treatment. On average, resistance to Sorafenib occurs at approximately 12.2 months, but can vary from months to years. In the lab, Sorafenib-resistant HCC cell lines usually are established in about 12 weeks. Several methods of to prevent Sorafenib resistance have been tried, including combinational treatments with: anti-EGFR antibodies (Cetuximab), cytotoxic chemotherapeutic drugs (Epirubicin, Cisplatin, 5-FU and Capecitabine), and immunotherapeutic drugs (anti-PD-1 antibodies). However, these combinational therapies have severe adverse side-effects limiting their effectiveness.

### Cholesterol Buildup and Cancer

Cholesterol buildup has been linked to oncogenesis and progression. In the case of HCC, non-alcoholic steatohepatitis, which includes a buildup of cholesterol in liver cells, can lead to HCC. Cholesterol buildup in mitochondria also appears to play a role in cancer. For example, expression of steroidogenic acute regulatory protein, or StAR protein, increases several fold in response to acute liver injury and chronic liver disease, such as NASH. StAR aids in transporting cholesterol from the outer mitochondrial membrane (OMM) to the inner mitochondrial membrane (IMM).

Aberrant accumulation of mitochondrial cholesterol has been described in solid tumors, such as in rats bearing transplanted Morris hepatomas. In the 1960s, Dr. Harold P. Morris used several chemicals known to cause liver cancer in rats. Soon after, the first Morris hepatomas (#3683, 3924A, 5123) were studied by several labs and, over the next 18 years, Dr. Morris developed and transplanted numerous strains of hepatomas. Morris hepatomas are rat liver cancers that were generated in laboratory settings using known chemical carcinogens. Liver cancer describes many different types liver carcinoma, while HCC is the most common form of primary liver cancer in adults. Most cases of HCC result from infections with hepatitis B or C, or cirrhosis of the liver caused by NASH or alcoholism. Morris tumors provide a model system for the study or liver cancer, including HCC

Moreover, the levels of mitochondrial cholesterol correlate with the degree of tumor growth and malignancy. In the case of HCC, mitochondrial cholesterol accumulation has been seen to correlate with enhanced StAR expression. However, in colon cancer, mitochondrial cholesterol accumulation is inversely correlated with ABCA1 expression. ABCA1 aids in pumping cholesterol out of the cell.

Mitochondrial accumulation of cholesterol in hepatocytes, in NASH, results in decreasing membrane fluidity and similar results have been reported in HCC. Consequently, specific mitochondrial carriers become defective, leading to altered transport rates and equilibria between the mitochondrial matrix and the cytosol. For example, glutathione (abbreviated GSH) is synthesized in the cytosol and transported into mitochondria by 2-oxoglutarate carrier (OGC; SLC25A11). Inhibition of SLC25A11, by excess cholesterol accumulation, leads to the depletion of mitochondrial GSH (mGSH) in primary cells and makes the cells more sensitive to oxidative stress-mediated cell death. Paradoxically, increased mitochondrial cholesterol accumulation in HCC does not lead to mGSH depletion due to the overexpression of SLC25A11 that overcomes the inhibitory effect of cholesterol. This adaptive response of mitochondria in HCC cancer cells to increased mitochondrial cholesterol while maintaining mGSH levels is advantageous for tumor growth.

Cholesterol can protect cancer cells from entering the apoptotic pathway. In addition, cholesterol also promotes tumor growth by increasing antioxidant defense. Antioxidants are becoming recognized for their ability to accelerate cell and tumor growth, for example in lung cancer progression and melanoma metastasis. In these tumors, increased expression of SLC25A11 in non-small lung cancer and melanoma facilitates NADPH supply to maintain mitochondrial function. In HCC cells, SLC25A11 promotes cell growth by sustaining ATP generation through oxidative phosphorylation and glycolysis. This ability of cancer cells to maintain unrestricted oxidative phosphorylation despite increased cholesterol trafficking is remarkable given that cholesterol accumulation in mitochondria from primary hepatocytes results in defective assembly of respiratory chain super-complexes.

Mitochondria from cells of various cancer types exhibit increased cholesterol accumulation. This mitochondrial cholesterol accumulation may account for mitochondrial dysfunction and play a role in the effectiveness of chemotherapeutics. Overexpression of SLC25A11 in cancer cells may help promote cancer cell growth by helping maintain mitochondrial ATP synthesis, increasing resistance to mitochondrial membrane permeabilization, and increasing resistance to chemotherapy.

### Statins

Statins inhibit the cholesterol synthesis pathway. Specifically, statins target, and inhibit, 3-hydroxy-3-methyl-glutaryl-coenzyme A reductase (HMG-CoA reductase, or HMGCR). The HMGCR enzyme is the rate-controlling enzyme of the mevalonate pathway, which produces cholesterol and other isoprenoids. Statins/HMGCR inhibitors form a class of lipid-lowering medications often prescribed to patients at high risk of cardiovascular disease. The targeted lipids, LDLs or low-density lipoproteins, are carriers of cholesterol, and therefore play a key role in the development of atherosclerosis and coronary heart disease.

Statin compounds resemble HMG-CoA and thus compete with binding of HMG-CoA to HMGCR. This competition effectively lowers the rate of the HMGCR enzyme, which, in turn, lowers the synthesis of LDL cholesterol. There are various forms of statins, under generic names atorvastatin, fluvastatin, lovastatin, pitavastatin, pravastatin, rosuvastatin, and simvastatin.

Statins may be useful in improving chemotherapeutics. For example, chemotherapeutics, when combined with statins, show improved efficacy in treating cancer. For example, fluvastatin has been shown to increase the effectiveness and cytotoxicity of the chemotherapeutic drug Sorafenib on melanoma cells. Also, lovastatin in combination with enzastaurin (a chemotherapeutic that suppresses angiogenesis) has been shown to inhibit HCC growth in vitro and in vivo. HCC growth has also been shown to be inhibited by the combination of celecoxib (a non-steroidal anti-inflammatory that showed promising results on preventing colon polyps) and statins.

The role of statins as anti-cancer treatments may be related to the elevated mitochondrial cholesterol levels in cancer cells discussed above. As previously noted, increased levels of mitochondrial cholesterol in cancer cells may result in those cells being more resistant chemotherapeutic agents, especially cell-death-inducing chemotherapeutics.

### Cholesterol Degrading Combination Therapies

Disclosed herein are compositions, treatments, and methods for treating, preventing, and reducing the risk of various diseases, disorders, and conditions. In some embodiments, the disease may be a disease or disorder, including metabolic-related disease and cancer. In some embodiments, the disease may be liver related, for example end-stage liver disease, non-alcoholic steatohepatitis, hepatocellular carcinoma. In other embodiments, the disease may be various cancers, such as melanoma, hepatocellular carcinoma, colon cancer, etc.

Disclosed herein are compositions, treatments, and methods for reducing resistance to chemotherapeutic treatments. In many embodiments, the disclosed compositions may be combined with chemotherapeutic compounds for treating cancer. In many embodiments, the disclosed compositions, methods, and treatments may enhance the susceptibility of one or more cancer cells to the therapeutic compound. In many embodiments, use of the disclosed compositions and methods may reduce the rate of resistance to the chemotherapeutic compound, and/or reduce the progression, growth, and/or recurrence of a cancer.

### Diseases and Conditions

Various diseases, conditions, and disorders may be treated with the disclosed compositions, compounds, and methods, for example metabolic-related diseases and cancers. In many embodiments, the diseases and conditions may be associated with alterations in cellular uptake, trafficking, accumulation, and/or metabolism of cholesterol, especially those that are associated with abnormal cell proliferation and/or survival. In many embodiments, the disease or condition may be a cancer. In some embodiments, the disease may be liver related, for example early, later, or end-stage liver disease, non-alcoholic fatty liver disease (NAFLD), non-alcoholic steatohepatitis (NASH), hepatocellular carcinoma. In other embodiments, the disease may be various diseases or conditions that may lead to one or more cancers, such as pancreatic cancer, melanoma, hepatocellular carcinoma, colon cancer, etc. In many embodiments, the disclosed diseases, conditions, and disorders may be one or more of intestinal carcinomas, adenocarcinoma, gastrointestinal carcinoid, pancreatic cancer, small cell carcinoma, leiomyosarcoma, and lymphomas.

The disclosed treatments may prevent or reduce progression of disease. In some embodiments, for example where the patient suffers from NASH, the disclosed treatments may prevent or reduce the risk of HCC development and end-stage liver disease.

### Tissues and Cells

Various tissues and cell types may be targeted with the disclosed compositions and methods. In one embodiment, the targeted cells may display abnormal cell proliferation and/or survival. In many embodiments, the tissue or cell type may be a cancerous tissue or cell. In various embodiments, the cells may be hepatic cells and various cells found in the liver. In other embodiments, the tissues may be one or more of skin cells, squamous cells, epithelial cells, epithelial cells of glandular origin, neuroendocrine cells, pancreatic cells, smooth muscle cells and lymphocytes, for example lymphocytes found in organs/walls of the gastrointestinal tract.

### Prevention of Chemotherapeutic Drug Resistance

The disclosed compounds, compositions, methods, and treatments may reduce or prevent cells from being resistant various chemotherapeutic compounds. In various embodiments, the chemotherapeutic compounds may be frontline systemic therapies, for example Sorafenib (Bayer), Lenvatinib (Merck), Regorafenib (Stivarga), pembrolizumab (Keytruda), Ramucirumab (Cyramza), Cabozantinib (Cometriq, Cabometyx).

The disclosed compounds, compositions, methods, and treatments may provide for sensitizing cells to various medicinal compounds. In some embodiments, the medicinal compounds may be one or more of chemotherapeutic or immuno-based therapies. In some embodiments the medicinal compounds may be Sorafenib (Bayer), or Lenvatinib (Merck).

Tumor targeted delivery of CDP (mRNA or DNA) will prevent resistance to and sensitize cancer cells to anti-cancer therapies when administered alone or in combination.

### Apoptosis

Apoptosis is a fundamental mechanism of programmed cell death and the evasion of this process is sometimes referred to as a “Hallmark of Cancer”. Apoptosis can be in induced by several means and proceeds via two main pathways. The intrinsic pathway involves the B-cell lymphoma (Bcl)-2 protein family, mitochondrial permeability and apoptosome formation, while the extrinsic pathway involves death receptors, death inducing signaling complex and caspase activation.

### Intrinsic Pathway

Induction of the intrinsic pathway may occur in response to internal pro-apoptotic stimuli such as DNA damage.

In the early phase of apoptosis, DNA damage activates pro-apoptotic (i.e. apoptosis promoting) members of the Bcl-family (PUMA; p53 upregulated modulator of apoptosis, BAD; Bcl-2-associated agonist of cell death and Noxa), which lead to the inhibition of anti-apoptotic members, Bcl-xL and Bcl-2. This, in turn, allows pro-apoptotic BAK (Bcl-2 homologous antagonist killer) and BAX (Bcl-2-associated x protein) to insert into the mitochondrial membrane and induce the loss of mitochondrial membrane integrity, which irreversibly commits the cell to apoptosis.

During the mid-phase of apoptosis, the release of Cytochrome C and other proteins from the mitochondria, together with pro-caspase-9, form the apoptosome in the cytoplasm. This leads to the activation of caspase-9 and, consequently, to the activation of the effector caspases, caspase-3/7. These effector caspases initiate key apoptotic events including the exposure of phosphatidylserine to the extracellular side of the cell membrane.

### Extrinsic Pathway

The extrinsic pathway is activated by the binding of ligands to members of the TNF receptor super family including CD120a, CD120b, CD95/FAS, Death Receptor (DR) 3, CD261/DR4, CD262/DR5, CD266 and CD358/DR6. The main apoptosis-inducing ligands are TNF-□, lymphotoxin-□, FasL/CD178 and TRAIL (TNF-related apoptosis-inducing ligand).

Following ligand binding to death receptors, downstream signaling pathways activate the formation of complexes such as the Death Inducing Signaling Complex (DISC). Ultimately, the early phase of this pathway is the activation of the initiator caspase, caspase-8. Within this early phase, there is potential crosstalk with the intrinsic pathway via caspase-8 mediated cleavage and activation of BID (BH3-interacting domain death agonist) into the active 15 kDa truncated form, tBID.

During the mid phase of apoptosis, caspase-8 activates the effectors caspases, caspase-3 and caspase-7, which in turn initiate key apoptotic events such as the exposure of phosphatidylserine to the extracellular side of the cell membrane. This is the point where the extrinsic and intrinsic apoptotic pathways converge.

In the late phase of apoptosis, the extrinsic and intrinsic pathways have converged. It begins with the activation of caspase-3/7 and results in DNA fragmentation and cell membrane disruption/blebbing. Briefly, caspase-3/7 release DFF40 from its inhibitor DFF45, allowing DFF40 to participate in DNA fragmentation. Mitochondrial Endonuclease G also contributes to DNA fragmentation. Caspase-3/7 activates ROCK1 which causes actomyosin-dependent membrane blebbing.

In many embodiments, apoptosis may be identified or assayed by analyzing cells for the presence, localization, activation/inhibition, expression, or changes therein, of any one or more of caspase 3, caspase 7, caspase 8, caspase 9, BID, tBID, Cytochrome C, pro-caspase 9, PUMA, BAD, Noxa, Bcl-xL, Bcl-2, BAK, BAX, and other factors and events well known to those of skill in the art.

### Cholesterol Degrading Proteins

Disclosed herein are various cholesterol, and cholesterol-related genes and proteins. In some embodiments, the disclosed genes and proteins may aid in transport and/or catabolism of cholesterol. In most embodiments the disclosed genes and proteins may be expressed in and/or targeted to the mitochondria. In many embodiments the disclosed genes and proteins may allow cells to degrade and/or catabolize cholesterol to ring opening, whereupon endogenous proteins and enzymes may further metabolize the molecule.

The disclosed compositions may include one or more genes and/or proteins that function aid in the flow of cholesterol into the mitochondrial matrix. In many embodiments, the compositions may include molecules that encode or include at least a portion of the steroidogenic acute regulatory (StAR) protein. StAR proteins for use with the present compositions and methods may include various forms of the StAR protein sequence, for example forms that may include one or more mutations, truncations, deletions, duplications, fusions, etc. or the disclosed proteins may be wild-type or native StAR proteins. In some embodiments, the disclosed StAR proteins may be modified to reduce transit time through the outer membrane, which may help to enhance mitochondrial import of cholesterol.

The disclosed compositions may include one or more genes and/or proteins that aid in catabolizing/degrading cholesterol. In most embodiments, the genes and/or proteins aid in catabolizing cholesterol within a mitochondrion. In many embodiments, the disclosed genes and/or proteins may be selected from cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx), and combinations thereof.

One or more of the disclosed genes, proteins, and enzymes may be packaged into one or more vector, construct or cassette. In various embodiment, a cassette that includes one or more cholesterol degrading enzymes may be referred to as a cholesterol catabolizing cassette (CCC). In some embodiments the cassette may be a construct and may include a nucleic acid sequence that codes for a protein that is about 80% or more identical to a protein coded for by any of the disclosed genes.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The terms “prevent” and “prevention” as used herein mean avoidance of the occurrence or of the re-occurrence of a disease as specified herein, by the administration of an antibody construct according to the invention to a subject in need thereof.

The terms “treat,” “treating,” and “treatment” refer to eliminating, reducing, suppressing, or ameliorating, either temporarily or permanently, either partially or completely, a clinical symptom, manifestation, or progression of an event, disease, or condition described herein. As is recognized in the pertinent field, methods, and drugs employed as therapies may reduce the severity of a given disease state, but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, hepatocellular carcinoma, etc. and/or reducing the number or severity of associated symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic treatment in an amount, duration, and repetition sufficient to induce a sustained improvement over pre-existing conditions, or a baseline indicator that reflects the severity of the hepatocellular carcinoma.

As disclosed herein, administration of the disclosed compounds and compositions may be via various routes. In some embodiments, the administration may be “Intravenous”—that is into a vein of a patient, e.g. by infusion (slow therapeutic introduction into the vein). In some embodiments, the administration may be “subcutaneous”—that is beneath the skin of the patient. In some embodiments, the administration may be by “infusion” or “infusing” wherein a solution is delivered to the patient through a vein for therapeutic purposes. Generally, this is achieved via an intravenous (IV) bag—a bag that can hold a solution which can be administered via the vein of a patient. In one embodiment, the IV bag is formed from polyolefin or polyvinyl chloride. In various embodiments, the disclosed compounds and compositions may be “co-administering”—that is two or more compounds or compositions may be administered during the same administration even, rather than sequentially. Generally, this will involve combining the two (or more) compounds or compositions into the same IV bag prior to co-administration thereof.

As disclosed herein “patient” or subject” includes an animal, such as a human, cow, horse, sheep, lamb, pig, chicken, turkey, quail, cat, dog, mouse, rat, rabbit or guinea pig. The animal can be a mammal such as a non-primate or a primate (e.g., monkey and human). In one embodiment, a patient is a human, such as a human infant, child, adolescent, or adult.

### EXAMPLES

### Example 1 Effect of Cholesterol Degradation on Growth in Response to Treatment with Sorafenib

Human Hep3B hepatocellular carcinoma cells (from ATCC at www.atcc.org/products/all/HB-8064.aspx) were grown in Eagle's minimum essential media (EMEM) supplemented with 10% heat-inactivated FBS, PenStrep (100 units/mL//100 mg) and 2mM L-Glutamine.

Cells would be transduced with lentiviral vectors containing an antibiotic resistance (here puromycin) gene. Thus, the optimal condition for selection in puromycin of the Hep3B cells was first established by subjecting un-transduced Hep3B cells to increasing concentrations of the antibiotic. The minimum concentration for selection was identified by determining the lowest concentration of puromycin needed to kill all cells over the course of 7 days.

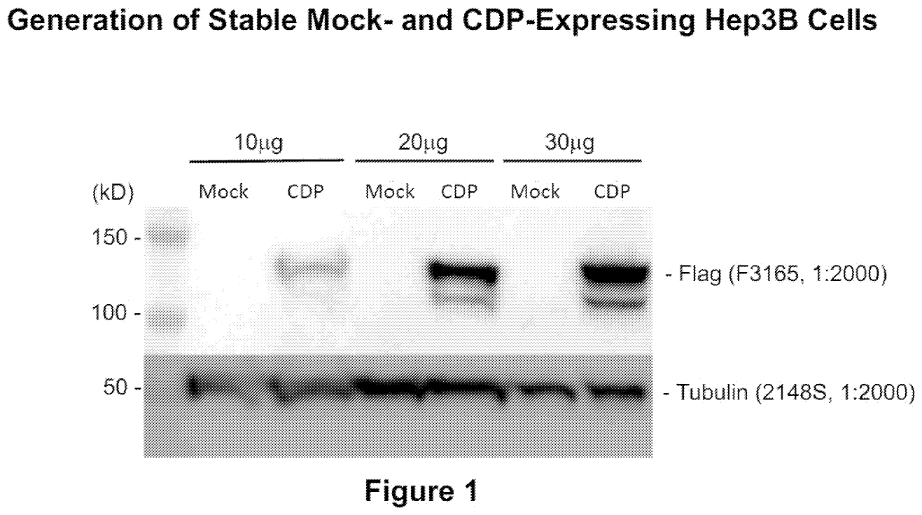

Hep3B cells were transduced with either empty lentivirus (MOI=5) or lentivirus harboring CDP (MOI=5). Transduced cells were selected by growth in puromycin (4 μg/mL) to establish stable cell lines of Mock-Hep3B (empty lentivirus) and CDP-Hep3B (lentivirus containing CDP). FIG. 1 is a representative western blot showing the expression of Flag-tagged CDP in CDP-Hep3B cells (“CDP”) and not in Mock-Hep3B cells (“Mock”). Briefly, total proteins were extracted from each stable cell line (Mock and CDP) using RIPA buffer and 10, 20 and 30 μg protein from each cell line was separated by SDS-PAGE, transferred onto a PVDF membrane, and probed with mouse anti-Flag antibody (Sigma, F3165, 1:2000) overnight. The specific bands were visualized using chemiluminescence and band intensities normalized to α/β-tubulin (anti-tubulin antibody; Santa Cruz, 2148S, 1:2000) to control for loading and transfer.

The growth and viability of Mock- and CDP-Hep3B cells were assessed by cell proliferation assay, using the tetrazolium salt 3-[4,5-di-methylthiazol-2yl]-2,5-diphenyl-tetrazolium bromide (MTT) reagent (resources.rndsystems.com/pdfs/datasheets/4890-025-k.pdf). Briefly, cells were cultured in 100 μL of culture medium in a 96-well flat-bottomed tissue culture plate. To determine the linearity of the assay, each stable cell line was plated at increasing densities (1500, 3000, 6000 and 12000 cells/well) and incubated for 24, 48 and 72 hours. The MTT reagent was added at each time point and cells were incubated for 4 hours to allow for intracellular reduction of the soluble yellow MTT reagent into the insoluble purple formazan dye. Detergent reagent was then added to each well to solubilize the formazan dye prior to measuring the absorbance of each sample in a microplate reader at 570 nm. Results indicated that the assay remained in the linear range throughout the first 24- and 48-hour periods.

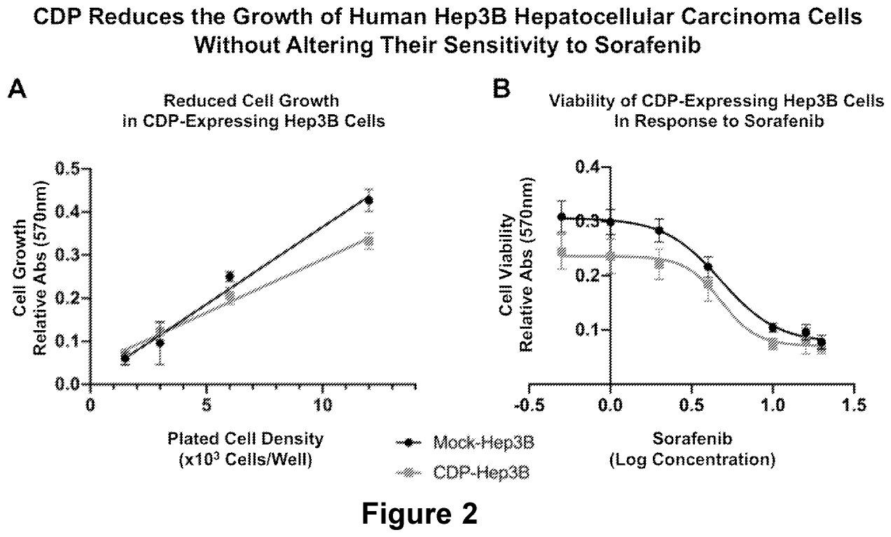

Growth rates were graphed for Mock-Hep3B and CDP-Hep3B cell lines at varying densities over 48 hours. FIG. 2A is a graph of Cell Growth versus plating density, to obtain rates of growth for CDP-Hep3B and Mock-Hep3B cells over 48 hours. This graph demonstrated that expression of CDP in the CDP-Hep3B cells resulted in a reduction in the growth rate (slope=0.0247 vs 0.0358 respectively, 2-way ANOVA P<0.0001, average of n=2 experiments).

Growth rates of stable cell lines were also obtained in the presence of Sorafenib. FIG. 2B is a representative graph of cell Viability versus Sorafenib concentration. These studies showed a similar reduction in the viability of CDP-Hep3B and Mock-Hep3B cells in response to the frontline therapy for HCC patients and multi-kinase inhibitor, sorafenib. Briefly, Mock- and CDP-Hep3B cells were plated at 12000 cells/well in a 96-well plate and exposed to increasing doses of sorafenib (0, 0.5, 1, 2, 4, 10 16, and 20 μM). Cell viability was assessed using the MTT assay after 48 hours as described above.

The expression of CDP in human Hep3B cells did not appear to significantly affect IC₅₀ in the presence of sorafenib as evidenced by fitting the data of FIG. 2B (calculated IC₅₀ values: 4.64±0.16 vs 4.76±0.62 for Mock-Hep3B, n=3 independent experiments).

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

1. A method of treating or preventing hepatocellular carcinoma in a subject in need thereof comprising:
expressing one or more cholesterol catabolizing protein in at least one liver cell of the subject;
reducing a growth rate of the at least one liver cell.

2. The method of claim 1, wherein the one or more cholesterol catabolizing protein is expressed in a mitochondrion of the liver cell.

3. The method of claim 2, wherein the at least one liver cell is contacted with at least one chemotherapeutic compound, and the chemotherapeutic is administered to the subject before, after, or while the cell is expressing the one or more cholesterol catabolizing protein.

4. The method of claim 3, wherein the one or more catabolizing proteins are selected from steroidogenic acute regulatory (StAR) protein, cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx), and combinations thereof.

5. The method of claim 4, wherein the chemotherapeutic compound is selected from Sorafenib and Lenvatinib.

6. The method of claim 5, wherein the method prevents or reduces a likelihood of a mammalian cell being resistant to chemotherapy.

7. The method of claim 6, wherein the liver cell enters the apoptotic pathway after expressing the one or more cholesterol catabolizing protein.

8. A method of treating or preventing hepatocellular carcinoma in a subject in need thereof comprising:
administering a composition to the subject, wherein the composition comprises one or more compounds that support expression of at least one cholesterol catabolizing protein in a mitochondrion of a mammalian cell.

9. The method of claim 8, wherein the subject is administered a chemotherapeutic compound.

10. The method of claim 9, wherein the chemotherapeutic compound is administered to the subject before, after, or with the composition.

11. The method of claim 10, wherein the at least one cholesterol catabolizing proteins are selected from steroidogenic acute regulatory (StAR) protein, cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx), and combinations thereof.

12. The method of claim 11, wherein the chemotherapeutic compound is selected from Sorafenib and Lenvatinib.

13. The method of claim 12, wherein the method prevents or reduces a likelihood of a liver cell being resistant to chemotherapy.

14. The method of claim 13, wherein the liver cell enters the apoptotic pathway after expressing the at least one cholesterol catabolizing protein.

15. A composition for reducing growth rate or viability of a hepatic carcinoma comprising:
a nucleic acid coding for at least one cholesterol catabolizing protein; and
a nucleic acid comprising a promoter that is active in a mammalian liver cell.

16. The composition of claim 15, wherein the nucleic acid is within a cell.

17. The composition of claim 15, wherein the nucleic acid is within a viral particle.

18. The composition of claim 15, wherein the nucleic acid is within a lipid nanoparticle.

19. The composition of claim 18, comprising a pharmaceutically acceptable compound or salt.

20. (canceled)

21. The composition of claim 19, wherein the at least one cholesterol catabolizing proteins are selected from steroidogenic acute regulatory (StAR) protein, cholesterol dehydrogenase (CholD), 3-ketosteroid Δ1-dehydrogenase (Δ1-KstD), anoxic cholesterol metabolism B enzyme (acmB), 3-ketosteroid 9α-hydroxylase (KshAB), 3β-hydroxysteroid dehydrogenase 2 (HSD2), P450-ferredoxin reductase-ferredoxin fusion protein (P450-FdxR-Fdx), and combinations thereof.

22-39. (canceled)


SEQUENCE LISTING (6 sequences)

SEQ ID NO: 1 - protein, 1122 aa, Artificial Sequence
  Description of Artificial Sequence Synthetic polypeptide
MLAKGLPPRSVLVKGCQTFLSAPREGLGRLRVPTGEGAGISTRSPRPFNEIPSPGDNGWLNLYHFWRETGTHKVHLHHVQNFQKYGPIYREKLGNVESVYVIDPEDVALLFKSEGPNPERFLIPPWVAYHQYYQRPIGVLLKKSAAWKKDRVALNQEVMAPEATKNFLPLLDAVSRDFVSVLHRRIKKAGSGNYSGDISDDLFRFAFESITNVIFGERQGMLEEVVNPEAQRFIDAIYQMFHTSVPMLNLPPDLFRLFRTKTWKDHVAAWDVIFSKADIYTQNFYWELRQKGSVHHDYRGILYRLLGDSKMSFEDIKANVTEMLAGGVDTTSMTLQWHLYEMARNLKVQDMLRAEVLAARHQAQGDMATMLQLVPLLKASIKETLRLHPISVTLQRYLVNDLVLRDYMIPAKTLVQVAIYALGREPTFFFDPENFDPTRWLSKDKNITYFRNLGFGWGVRQCLGRRIAELEMTIFLINMLENFRVEIQHLSDVGTTFNLILMPEKPISFTFWPFNQEATQQTDGTSSTQEKTPQICVVGSGPAGFYTAQHLLKHPQAHVDIYEKQPVPFGLVRFGVAPDHPEVKNVINTFTQTAHSGRCAFWGNVEVGRDVTVPELREAYHAVVLSYGAEDHRALEIPGEELPGVCSARAFVGWYNGLPENQELEPDLSCDTAVILGQGNVALDVARILLTPPEHLERTDITKAALGVLRQSRVKTVWLVGRRGPLQVAFTIKELREMIQLPGARPILDPVDFLGLQDKIKEVPRPRKRLTELLLRTATEKPGPAEAARQASASRAWGLRFFRSPQQVLPSPDGRRAAGVRLAVTRLEGVDEATRAVPTGDMEDLPCGLVLSSIGYKSRPVDPSVPFDSKLGVIPNVEGRVMDVPGLYCSGWVKRGPTGVIATTMTDSFLTGQMLLQDLKAGLLPSGPRPGYAAIQALLSSRGVRPVSFSDWEKLDAEEVARGQGTGKPREKLVDPQEMLRLLGHTDGASSSSEDKITVHFINRDGETLTTKGKVGDSLLDVVVENNLDIDGFGACEGTLACSTCHLIFEDHIYEKLDAITDEENDMLDLAYGLTDRSRLGCQICLTKSMDNMTVRVPETVADARQSIDVGKTSDYKDDDDK

SEQ ID NO: 2 - protein, 427 aa, Unknown
  Description of Unknown Cholesterol dehydrogenase sequence
MPTLYKKAGFQLGSLENLYFQGHHHHHHGTMAGFLNCCPGCCMEPGASDYKDDDDKMLRRMGDASLTTELGRVLVTGGAGFVGANLVTTLLDRGHWVRSFDRAPSLLPAHPQLEVLQGDITDADVCAAAVDGIDTIFHTAAIIELMGGASVTDEYRQRSFAVNVGGTENLLHAGQRAGVQRFVYTSSNSVVMGGQNIAGGDETLPYTDRFNDLYTETKVVAERFVLAQNGVDGMLTCAIRPSGIWGNGDQTMFRKLFESVLKGHVKVLVGRKSARLDNSYVHNLIHGFILAAAHLVPDGTAPGQAYFINDAEPINMFEFARPVLEACGQRWPKMRISGPAVRWVMTGWQRLHFRFGFPAPLLEPLAVERLYLDNYFSIAKARRDLGYEPLFTTQQALTECLPYYVSLFEQMKNEARAEKTAATVKPG

SEQ ID NO: 3 - protein, 510 aa, Unknown
  Description of Unknown 3-ketosteroid delta1-dehydrogenase sequence
MQDWTSECDVLVVGSGGGALTGAYTAAAQGLTTIVLEKTDRFGGTSAYSGASIWLPGTQVQERAGLPDSTENARTYLRALLGDAESERQDAYVETAPAVVALLEQNPNIEFEFRAFPDYYKAEGRMDTGRSINPLDLDPADIGDLAGKVRPELDQDRTGQDHAPGPMIGGRALIGRLLAAVQSTGKAELRTESVLTSLIVEDGRVVGAEVESGGETQRIKANRGVLMAAGGIEGNAEMREQAGTPGKAIWSMGPFGANTGDAISAGIAVGGATALLDQAWFCPGVEQPDGSAAFMVGVRGGLVVDSAGERYLNESLPYDQFGRAMDAHDDNGSAVPSFMIFDSREGGGLPAICIPNTAPAKHLEAGTWVGADTLEELAAKTGLPADALRSTVEKFNDAAKLGVDEEFHRGEDPYDAFFCPPNGGANAALTAIENGPFYAARIVLSDLGTKGGLVTDVNGRVLRADGSAIDGLYAAGNTSASLSGRFYPGPGVPLGTAMVFSYRAAQDMAK

SEQ ID NO: 4 - protein, 595 aa, Unknown
  Description of Unknown Anoxic cholesterol metabolism beta enzyme sequence
MPTLYKKAGFQLGSGTENLYFQGTMSIETNTYDVIVVGSGAGAMLAAARAHDLGLSVLVVEKSDKYGGTSAVSGGAVWIPNNSQMQIKDSFDEALTYLKAATQGLVAEDRLLAYLESAPQMVEYINANMTLQYFPCHRYPDYYQHLPGAKPGGRTMEPMLFDAALLGDEFANLRMAYTGTLLMGKASMTATEAHVMLAKEPGWMLQVIKSLGRYYLDLPWRLKSRHDRKRGLGNAMAAGLRHALLERKVPLWLNTPFESLITEGAENKRVTGIVVKRNGQTLQLTARRGVVLGAGGFERNQQMREQYLPKPTNAAWSATPPHNTGDTIRAAMDIGARAELMDWAWWVPSIHVPGEAAQTGLFAERNLPGCIVVNGKGQRFINEASPYLEFGAAMYENHARSGSAVPAWLIFDGKFRYNYPMGPLMPGQIQPDRKAWLGKVYWRDDTLEGLAKQIGVDAAGLKQSVELNNQYAQDGKDREFDKGGNVFDRYYGDYNVKPNPCLAPIGKPPYYAMRVDAGDIGTKGGLLTDKDARVLDESDRPIEGLYCIGNNSASVMGKAYPGAGGTLGPAMTFGFRAANHIAASKYPYDVPDYAG

SEQ ID NO: 5 - protein, 860 aa, Unknown
  Description of Unknown 3-ketosteroid 9alpha-hydroxylase sequence
MAPYSLLVTRLQKALGVRQYHVASVLCQRAKVAMSDYKDDDDKGGGGSMSIDTARSGSDDDVEIREIQAAAAPTRFARGWHCLGLLRDFQDGKPHSIEAFGTKLVVFADSKGQLNVLDAYCRHMGGDLSRGEVKGDSIACPFHDWRWNGKGKCTDIPYARRVPPIAKTRAWTTLERNGQLYVWNDPQGNPPPEDVTIPEIAGYGTDEWTDWSWKSLRIKGSHCREIVDNVVDMAHFFYIHYSFPRYFKNVFEGHTATQYMHSTGREDVISGTNYDDPNAELRSEATYFGPSYMIDWLESDANGQTIETILINCHYPVSNNEFVLQYGAIVKKLPGVSDEIAAGMAEQFAEGVQLGFEQDVEIWKNKAPIDNPLLSEEDGPVYQLRRWYQQFYVDVEDITEDMTKRFEFEIDTTRAVASWQKEVAENLAKQAEGSTATPGSGATNFSLLKQAGDVEENPGPMAPYSLLVTRLQKALGVRQYHVASVLCQRAKVAMSGYPYDVPDYAGGGGSMTAVQAPVTSRATVLTVSAVVQETADAVSLVFDVPDDRREDFTYRPGQFLTLRIPSDRTGSVARCYSLASSPFTGEPPKVTVKRTAGGYGSNWLCDNIVAGRSIEVLPPAGVFTPADLTEKLVLFAGGSGITPVMSILESALHSGNRDVVLIYGNRDEKSVIFAEKLRELAARHAGALTVVHWLESVQGLPSPQQLATLISPFADHRAYMCGPGPFMDTVREGLLLAGVPKDRIHAEVFTSLSGDPFADVPLVEIDESDADATSATVQLDGEEHDLVWPRSATLVDVMLSKGLDVPYSCREGECGSCACTVVEGDVDSLPSAILDEEDIANGYVLACQARPKSDHVRIEF

SEQ ID NO: 6 - protein, 372 aa, Unknown
  Description of Unknown 3beta-hydroxysteroid dehydrogenase sequence
MGWSCLVTGAGGLLGQRIVRLLVEEKELKEIRALDKAFRPELREEFSKLQNRTKLTVLEGDILDEPFLKRACQDVSVVIHTACIIDVFGVTHRESIMNVNVKGTQLLLEACVQASVPVFIYTSSIEVAGPNSYKEIIQNGHEEEPLENTWPTPYPYSKKLAEKAVLAANGWNLKNGDTLYTCALRPTYIYGEGGPFLSASINEALNNNGILSSVGKFSTVNPVYVGNVAWAHILALRALRDPKKAPSVRGQFYYISDDTPHQSYDNLNYILSKEFGLRLDSRWSLPLTLMYWIGFLLEVVSFLLSPIYSYQPPFNRHTVTLSNSVFTFSYKKAQRDLAYKPLYSWEEAKQKTVEWVGSLVDRHKETLKSKTQ